(19) United States
(12) Patent Application Publication
(10) Pub. No.: US 2013/0149783 A1
(43) Pub. Date: Jun. 13, 2013

(54) CLEAVABLE MODIFICATIONS TO REDUCIBLE POLY (AMIDO ETHYLENIMINES)S TO ENHANCE NUCLEOTIDE DELIVERY

(71) Applicants: James William Yockman, West Jordan, UT (US); Jonathan H. Brumbach, Salt Lake City, UT (US); Sung Wan Kim, Salt Lake City, UT (US)

(72) Inventors: James William Yockman, West Jordan, UT (US); Jonathan H. Brumbach, Salt Lake City, UT (US); Sung Wan Kim, Salt Lake City, UT (US)

(21) Appl. No.: 13/635,479

(22) PCT Filed: Mar. 16, 2011
(86) PCT No.: PCT/US2011/028690

§ 371 Date: Feb. 13, 2013

Related U.S. Application Data
(60) Provisional application No. 61/314,469, filed on Mar. 16, 2010.

(51) Int. Cl. C07C 271/22

(52) U.S. Cl. CPC C07C 271/22
USPC 435/440; 560/148; 536/23.1; 252/182.3

(57) ABSTRACT

Polyplex formulations were prepared using p(TETA/CBA), its PEGylated analog, p(TETA/CBA)-g-PEG2k, and mixtures of the two species at 10/90 and 50/50 wt %, respectively. Increasing PEG wt % inhibited polyplex formation. This work demonstrates the feasibility of preparing homogenous polyplexes by altering the PEG wt % using a mixture of p(TETA/CBA) and p(TETA/CBA)-g-PEG2k products. Further, a single-step method of making p(TETA/CBA)-g-PEG2k is disclosed.

Gene therapy is a feasible alternative for treating genetically based diseases that conventional therapies currently manage. However, its clinical success is hampered by unclear design and formulation requirements to develop safe and efficient nucleic acid carriers. Recent research advancements have improved carrier safety and efficacy through carrier modifications to alter surface charge and/or tissue specificity using polyethylene glycol (PEG) and/or cell-specific targeting ligands (1). Polymeric non-viral gene carriers have distinct advantages because, if designed prudently, they are non-immunogenic and are easily modified to exhibit multi-functional properties (2). Non-viral polycations are also relatively cost-effective, easy to produce industrially and can carry large amounts of therapeutic nucleic acid (3,4).

Many structurally different polymers and copolymers consisting of linear, branched, or dendron architecture have been tested for their efficacy and suitability for in vitro and in vivo use. The polyethylenimine gene carriers (PEIs) have been most rigorously studied and are a standard in the field because they easily condense DNA into nucleic acid/polycation nanoparticles (polyplexes) that protect nucleic acid from serum nuclease degradation and exhibit relatively high transgene delivery and expression in many cell types in vitro and in vivo. Unfortunately, PEIs often exhibit cellular toxicity due to intracellular accumulation of non-degradable polycations (3, 5). Increased PEI molecular weight and branching, which influence polycation charge density, correlate with increased transgene expression, but also cellular toxicity. Conversely, low molecular weight PEIs show reduced cellular toxicity that correlate with reduced transgene expression (6,7). As predicted, the design of degradable gene carriers such as the poly(amidoamine) (SS-PAA), poly(amido ethylenimines) (SS-PAEI) and poly(b-amino ester) families have demonstrated comparable or improved activity and less cell toxicity when compared to PEIs (8, 9, 10). Reducible SS-PAEIs are synthetic analogs of the PEI family but with the aforementioned advantages (11). A recent abstract provided results showing hyperbranched, SS-PAAs can condense plasmid DNA (pDNA) into polyplexes with sizes similar to bPEI25kDa, and further functional studies were encouraged (12).

Often, cationic polyplexes interact with net negatively charged proteins found in serum, which often leads to particle aggregation and reduced efficacy in vitro and in vivo (13, 14, 15). To overcome this hurdle, poly(ethylene glycol) (PEG) conjugation to polycations has been employed, and studies have shown that pegylation often improves carrier function in the presence of serum. However, previous studies have also clearly shown that increasing targeting ligand and/or PEG conjugation to PEIs, especially low molecular weight (LMW) PEI (˜5 kDa), adversely effects polyplex formation and carrier function (16, 17).

To advance the understanding in the design and, more importantly, formulation of hyperbranched SS-PAEIs and their corresponding graft PEG copolymers, several SS-PAEI polycationic gene carriers were synthesized, and the influence of varying the PEG/polycation wt % on polyplex formation, size, surface charge, morphology, serum stability, and, ultimately, biological activity were studied and are described herein. Polyplex formulations to complex plasmid DNA or siRNA were prepared using a SS-PAEI, p(TETA/CBA), its PEGylated counterpart, p(TETA/CBA)-g-PEG2k, or mixtures of the two species at 10/90 and 50/50 wt/wt %, respectively. Altering the wt/wt % was employed to identify a suitable strategy to easily alter polyplex composition and identify a suitable formulation with synthetic ease.

An illustrative composition according to the present invention comprises a graft copolymer of poly(TETA/CBA) and polyethylene glycol.

Another illustrative embodiment of the present invention comprises a complex comprising a nucleic acid and a graft copolymer of poly(TETA/CBA) and polyethylene glycol. The nucleic acid can comprise plasmid DNA or siRNA, for example. The complex can further comprise poly(TETA/CBA) mixed with the graft copolymer.

Still another illustrative embodiment of the present invention comprises a mixture of poly(TETA/CBA) and a graft copolymer of poly(TETA/CBA) and polyethylene glycol.

Yet another illustrative embodiment of the invention comprises a method of transfecting a cell comprising contacting the cell with a complex comprising a nucleic acid and a graft copolymer of poly(TETA/CBA) and polyethylene glycol. The nucleic acid can comprise plasmid DNA or siRNA, for example. The complex can further comprise poly(TETA/CBA) mixed with the graft copolymer.

### BRIEF DESCRIPTION OF THE SEVERAL VIEWS OF THE DRAWINGS

Scheme 1 shows a schematic representation of synthesis of p(TETA/CBA)1k and p(TETA/CBA)5k according to the present invention.

Scheme 2 shows a scheme for synthesis of p(TETA/CBA)5k-g-PEG2k according to the present invention. Schematic representations of 100 wt % p(TETA/CBA)5k, 10/90 wt % p(TETA/CBA)5k-g-PEG2k, 50/50 wt % p(TETA/CBA)5k-g-PEG2k, 100 wt % p(TETA/CBA)5k-g-PEG2k, and SS-PAEI+PEG2k are also shown.

Scheme 3 shows a scheme for “single-step” synthesis of p(TETA/CBA)-g-PEG2k according to the present invention.

FIGS. 1A-D show transfection efficiencies (FIGS. 1A and 1B) and cell viabilities (FIGS. 1C and 1D) in SVR (FIGS. 1A and 1C) and HUVEC (FIGS. 1B and 1D) endothelial cells of different p(TETA/CBA) molecular weight analogs combined with pCMVLuc to form polyplexes, compared to a positive control (bPEI 25 kDa). Commercial bPEI polyplexes were prepared at N/P 10, and p(TETA/CBA) polyplexes were prepared at w/w 24.

FIGS. 2A and 2B show transfection efficiency and cellular viability, respectively, with different molecular weights of p(TETA/CBA).

FIG. 3 shows a comparison of p(TETA/CBA)5k/pCMVLuc transfection efficiency in the presence (checked bars) and absence (hatched bars) of 10% serum in culture media. Transfection efficiency was evaluated by luciferase transgene expression. p(TETA/CBA) exhibits greater reporter transgene expression than bPEI 25 kDa in serum containing media, but is still perturbed compared to its performance in the absence of serum.

FIGS. 4A and 4B, respectively, show particle size and zeta-potential measurements of p(TETA/CBA)5k (checked bars) and p(TETA/CBA)5k-g-PEG2k/pCMVLuc (hatched bars) polyplexes at increasing polymer concentrations using known amounts of pDNA.

FIG. 5A shows polyplex stability in 90% rabbit serum at 37° C. for p(TETA/CBA)5k, poly(TETA/CBA)5k-g-PEG2k, 10/90 (10% PEG) and 50/50 (50% PEG) wt/wt % formulations for p(TETA/CBA)5k-g-PEG2k and p(TETA/CBA)5k, respectively; 500 ng pCMVLuc was complexed with each formulation (w/w 24).

FIG. 5B shows the relative percent of intact pBLuc compared to the 0-hr control over time derived from pixel intensity: (⋄) control (free pDNA); (▪) p(TETA/CBA); (Δ) p(TETA/CBA)-PEG2 kDa (10%); (∇) p(TETA/CBA)-PEG2 kDa (50%); () p(TETA/CBA)-PEG2 kDa (100%).

FIGS. 6A-D, respectively, show p(TETA/CBA)5k, 10% PEG, 50% PEG, and p(TETA/CBA)-PEG2k polyplex formulations visualized with TEM.

FIG. 6E shows particle size (bars with small checks) and zeta potential (bars with large checks) of bPEI, p(TETA/CBA), 10% PEG, and 50% PEG.

FIG. 6F shows comparisons of p(TETA/CBA), 10% PEG, and 50% PEG polyplex sizes using TEM (bars with large checks) and dynamic light scattering (DSL; bars with small checks).

FIGS. 7A and 7B show transfection efficiency (FIG. 7A) and cell viability (FIG. 7B) of p(TETA/CBA)5k, 10/90, 50/50, and 0/100% p(TETA/CBA)5k/p(TETA/CBA)5k-g-PEG2k wt % polyplex formulations in the presence and absence of serum.

FIG. 8 shows particle sizes of nanocomplexes when the polymers are mixed at different percent weight ratios and with different weight/weight ratios of polymer(s) to siRNA.

FIGS. 9A-F show transfection efficiency of p(TETA/CBA)-g-PEG2k over a broad range of % weight and PEG formulations.

FIG. 10 shows increases in pegylation ratio decrease stability of complexes in 90% serum.

FIGS. 11A-C show biodistribution patterns of plasmid DNA after injection in mice as nanocomplexes with p(TETA/CBA)-g-PEG2k/p(TETA/CBA).

FIG. 12 shows mHIF-1a inhibition following intravenous or local subcutaneous injection of 55 μg of siRNA/p(TETA/CBA)-g-PEG.

### DETAILED DESCRIPTION

Before the present improvement to reducible poly(amido ethylenimine)s and methods are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference.

It must be noted that, as used in this specification and the appended claims, the singular forms “a,” “an,” and “the” include plural referents unless the context clearly dictates otherwise.

Unless defined otherwise, all technical and scientific terms used herein have the same meanings as commonly understood by one of ordinary skill in the art to which this invention belongs.

In describing and claiming the present invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.” As used herein, “consisting of” and grammatical equivalents thereof exclude any element, step, or ingredient not specified in the claim. As used herein, “consisting essentially of” and grammatical equivalents thereof limit the scope of a claim to the specified materials or steps and those that do not materially affect the basic and novel characteristic or characteristics of the claimed invention.

The clinical advancement of polycationic gene carriers is hampered by unclear design and formulation requirements. In the present work, it is shown that a graft copolymer of polyethylene glycol (PEG) and a branched SS-PAEI can be synthesized and used in conjunction with the polycationic SS-PAEI during formulation to alter the relative PEG wt %, thereby altering the physiochemical characteristics of the gene carrier population to easily study the design and formulation requirements to improve biological activity of gene carriers. Knowing that PEG and/or targeting ligand conjugation can interfere with polyplex formation and carrier function, this work demonstrates the feasibility of overcoming the problem and preparing homogenous polyplexes by altering the PEG wt % using a mixture of p(TETA/CBA) and p(TETA/CBA)-g-PEG2k products that are functionally viable.

In the present study, there was developed a novel gene carrier comprised of an efficient and non-toxic bioreducible polycation in conjunction with polyethylene glycol to improve carrier performance in the presence of serum. In addition, there is provided a feasible and facile approach to tailor polycationic-PEG copolymer formulations to alter PEG wt % and obtain optimal physiochemical properties for ideal gene carrier function. By doing so, synthesis of multiple copolymers for gene delivery can be avoided when designing a gene carrier with preferred physiochemical properties for in vitro use, which may also be employed for facile in vivo evaluation.

To reduce p(TETA/CBA) PDI following the uncontrolled Michael-addition of the bisacrylamide group with TETA, ultrafiltration was performed using a higher molecular weight cut-off membrane (5 kDa) than was used previously (11). As expected, this approach was effective in reducing the PDI and correlates with a relative increase in molecular weight. Because an increased polyethyleneimine molecular weight and branching profile has been shown to correlate with transgene expression and cellular toxicity, the present study investigated this putative effect with p(TETA/CBA) and found no significant influence on its biological activity in primary and immortalized endothelial cell lines (6,7). These results are explained by the gene carrier's ability to exploit the intracellular redox potential and avoid disruption of intracellular function by relatively high molecular weight polycationic species (21).

While p(TETA/CBA) demonstrated significantly better transgene expression than bPEI 25 kDa in serum-containing media, p(TETA/CBA) delivery capacity was noticeably lower when compared to its activity in the absence of serum. Therefore, to reduce p(TETA/CBA)/pDNA polyplex interactions with serum proteins and thus improve carrier function in the presence of serum, polyethylene glycol was conjugated to p(TETA/CBA)5k at an equimolar ratio and confirmed by ¹H NMR following purification. The corresponding relative molecular weight was also in agreement with what is expected for equimolar conjugation when analyzed using AKTA FPLC. Conjugating polyethylene glycol to p(TETA/CBA)5k reduced polyplex surface charge, however, it adversely affected nucleic acid condensation (16, 22). Because polyethylene glycol and/or ligand conjugation for cell-specific gene delivery commonly mitigates nucleic acid condensation, synthesis of multiple co-polymeric gene carriers is required to ascertain optimal ratios for maximal carrier performance. In an attempt to overcome this problem and avoid the need to synthesize multiple carriers for screening, this study investigated the feasibility of optimizing PEG/polycation wt % (or ratio) by formulating mixtures of a polycation and its corresponding pegylated counterpart. Polyplex stability in serum was evaluated in this study comprised of p(TETA/CBA)5k alone, p(TETA/CBA)5k-PEG2k alone, and 10/90 or 50/50 wt % of p(TETA/CBA)5k-PEG2k/p(TETA/CBA)5k, respectively. Polyplex formed using p(TETA/CBA) and 10/90% sufficiently protects up to 70% of the pDNA from serum nuclease degradation over 6 hr. Increasing the p(TETA/CBA)5k-PEG2k wt % to 50 and 100% reduced the relative pDNA protection in serum, which correlates with the capability of each formulation to condense pDNA into nano-sized polyplex using DLS and TEM.

Luciferase transgene expression and cell viability was investigated in cell culture using the aforementioned formulations to evaluate their bioactivity. Polyethylene glycol was able to improve gene delivery in serum-containing media compared to p(TETA/CBA) alone, however, this improvement was observed only at specific polyethylene glycol ratios. These results provide evidence that polyethylene glycol/polycation ratios can be altered to easily study and optimize polyethylene glycol ratios for improved carrier function and avoid synthesis of multiple bio-reducible co-polymers with different physiochemical characteristics currently employed for gene carrier optimization.

### Experiments and Protocols

### Materials and Methods

Materials.

Triethylenetetramine (TETA), tris(2-carboxyethyl)phosphine) (TCEP), ethylemaleimide (NEM), hyperbranched polyethylenimine (bPEI, Mw 25 000) and HPLC grade methanol were purchased from Sigma-Aldrich (St. Louis, Mo.). Cystamine bisacrylamide (CBA) was purchased from Polysciences, Inc. (Warrington, Pa.). Ultrafiltration devices and regenerated cellulose membranes (1 kDa, 5 kDa, and 10 kDa) were supplied by Millipore Corporation (Billerico, Mass.). The reporter gene plasmid pCMVLuc was constructed by insertion of luciferase cDNA into a pCI plasmid (Promega, Madison, Wis.) driven by the pCMV promoter and was purified using Maxiprep (Invitrogen, Carlsbad, Calif.) protocols. Dulbecco's Modified Eagle's Medium (DMEM), penicillin streptomycin, trypsin-like enzyme (TrypLE Express), and Dulbecco's phosphate buffered saline were purchased from Gibco BRL (Carlsbad, Calif.). EBM-2 with EGM-2 singlequots was purchased from Lonza (Basel, Switzerland). Fetal bovine serum (FBS) was purchased from Hyclone Laboratories (Logan, Utah).

Polymer Synthesis.

p(TETA/CBA).

Synthesis of p(TETA/CBA) was performed by a modification to the previously described method at 50° C. (1). The polymerization reaction was split in half after the pH was adjusted to 7.0 and purified using ultrafiltration and a 1 kDa or 5 kDa MWCO regenerated cellulose membrane and subsequently lyophilized. (Scheme 1).

p(TETA/CBA)5k-g-2k.

Methoxy PEG 2k was dried using anhydrous toluene and subsequently precipitated in anhydrous ice-cold ether. The white precipitate was collected and dried in vacuo. The mPEG2k was then activated using p-nitrophenyl chloroformate in DCM (dichloromethane) as solvent and reacted on ice overnight while being stirred. The activated PEG product was collected by precipitation in anhydrous ice-cold ether and dried in vacuo. Following NMR analysis to assess the degree of PEG activation, p(TETA/CBA)5k and equal molar active PEG2k were dissolved in anhydrous pyridine/DMSO as solvent and the poly(ethylene glycol)-carbonate solution was added drop wise to the dissolved p(TETA/CBA)5k. The reaction was stirred at room temperature and monitored at 400 nm with UV-VIZ. When the reaction was complete around 16 hrs. The sample was purified by ultrafiltration (5 kDa MWCO) before being lyophilized. Conjugations using PEG5k and PEG10k were also performed similarly, however, they were purified using 10 or 20 kDa MWCO regenerated cellulose membranes, respectively, before being lyophilized. The composition of poly(TETA/CBA)-g-PEG copolymer conjugates was monitored by NMR to evaluate the relative amount of PEG conjugation by integrating appropriate peak area under the curve (AUC). ¹H NMR spectra were obtained on a Varian Inova 400 MHZ NMR spectrometer (Varian, Palo Alto, Calif.) using standard proton parameters. Chemical shifts were referenced to the residual H₂O resonance at approximately 4.7 ppm.

Polymer Characteristics.

Relative molecular weight analysis was performed on p(TETA/CBA)1k, p(TETA/CBA)5k, and p(TETA/CBA)5k-PEG2k by AKTA/FPLC (Amersham Pharmacia Biotech Inc.). A SuperdexPeptide column HR 10/30 was used to analyze p(TETA/CBA)1k (2 mg/mL). The eluent buffer (0.3 M NaAc, pH 4.4) with 30% (v/v) acetyl nitrile eluent was filtered through a 0.2 mm filter (Nylon, Alltech) and degassed prior to use. Flow rate was set at 0.4 mL/min. The calibration curve was prepared using poly(hydroxypropyl methacrylic acid) (poly(HMPA)) standards ranging from 2 kDa to 10 kDa. p(TETA/CBA)5k and p(TETA/CBA)5k-PEG2k were analyzed under the same conditions as above but using a Superose 6 10/300 GL column and poly(HMPA) standards ranging from 40 kDa to 150 kDa.

Polycation Branching.

Relative degree of branching was determined as previously described by the reduction and protection of disulfide bonds using Tris(2-carboxyethyl)phosphine hydrochloride (TCEP) and N-ethylmaleimide (NEM), respectively (5). MALDI-TOF analysis was performed on the polymer repeat unit NEM conjugates. MALDI-TOF analysis was performed on a Voyager-DE STR Biospectrometry Workstation (PerSeptive Biosystems) in positive-ion mode with delayed extraction. Spectra were externally calibrated using a peptide standard mixture spanning a nominal mass range from 325 to 2465.

Acid-Base Titrations.

The buffering capacity of each polycation was determined using a previously established method (14). In brief, 6 mg polymer was dissolved in 30 mL NaCl solution (0.1 M) and was initially titrated to pH 10 with 0.1M NaOH. The pH was then lowered with the addition of 0.1 M HCl. Because the absolute molecular weight is not know for these polymers, titration values are presented as mmol HCl required to lower the pH of the polycation solution from 7.4-5.1, and bPEIk 25 kDa was used as a reference control.

Light Scattering and z-Potential Measurements.

The surface charge and polymer/pDNA particle (polyplex) diameters were measured at 25° C. using a Zetasizer 2000 instrument (DTS5001 cell) and a dynamic light scattering (DLS) unit on a Malvern 4700 system, respectively. Polyplexes were prepared by adding equal volume polymer solution (200 ml) at increasing concentrations in HEPES buffer (20 mM, pH 7.4, 5% glucose) with a desired concentration of 15 mg pDNA in HEPES buffer (200 ml). Polyplexes were allowed to equilibrate for 30 min. and were subsequently diluted in filtered milliQ water to a final 2 mL volume.

Transmission Electron Microscopy (TEM).

Polyplexes were prepared in HEPES buffer (20 mM, pH 7.4, 5% glucose) at 0.05 mg/ml and 5 ml was deposited on TEM copper grid plates to dry. Residual buffer salt was removed by carefully rinsing each grid with filtered deionized water thrice. The samples were then stained with filtered phosphotungstenic acid (PTA) for 1 min before washing again with filtered deionized water. Images were visualized using a Technai T12 scope (EFM) at 80 kV. Magnification ranging from 20,000 to 200,000× was utilized and the micrograph images were taken at 110,000×.

Polyplex Stability in 90% Fresh Rabbit Serum.

Polyplex stability in serum was evaluated using an optimized protocol. In brief, 500 ng free pDNA or polymer/pDNA polyplexes were formed in HEPES buffer by mixing solutions of equal volume at a polymer/pDNA weight-to-weight (w/w) of 24 and allowed to equilibrate for 30 min. Preformed polyplexes were then diluted in 90% fresh rabbit serum and incubated at 37° C. over time. 25 ml aliquots (125 ng pDNA) were taken at each time point and 10 ml stop buffer (250 mM NaCl, 25 mM EDTA, 2% SDS) was added to each. The samples were frozen at −70° C. until further analysis. Once the samples were thawed, they were incubated overnight at 60° C. to completely dissociate polycations from the pDNA, and 2 ml of 50 mM DTT was added to each sample and incubated at 37° C. for an additional 30 min to ensure complete decomplexation. Lastly, the samples were loaded onto a 2% agarose gel stained with ethidium bromide (EtBr) and subjected to electrophoresis at 96 V for 30 min in TAE (40 mM Tris-acetate, 1 mM EDTA) buffer. The gel image was viewed using GelDoc software.

Cell Culture.

Mouse pancreatic islet endothelial cells (SVR) and colon adenocarcinoma cells (CT-26) (ATCC, Manasses, Va.) were cultured in DMEM containing 10% FBS and 1% penicillin-streptomycin at 37° C. in a humidified incubator with an atmosphere containing 5% (v/v) CO₂. Human Umbilical Vein Endothelial Cells (HUVEC) (Invitrogen) were cultured in EBM-2 with EGM-2 singlequots media at 37° C. in a humidified incubator with an atmosphere containing 5% (v/v) CO₂.

In Vitro Transgene Expression.

Luciferase reporter gene expression in cell culture was performed using each polymer and pCMVLuc plasmid DNA. Cells were plated in 24-well plates containing 0.5 mL of medium. Once cell confluency reached 70%, polyplexes were prepared using 0.5 mg pDNA at weight-to-weight (w/w) ratios equal to 24 in HEPES Buffer. Polyplexes were allowed to equilibrate for 30 min and the cells were transfected in the presence of serum. 20 ml polyplex (0.5 mg pDNA) was added to each well and allowed to incubate for 4 hrs. The culture medium was replaced with fresh serum-containing medium and the cells remained in the incubator for a total of 48 h. Cells were then washed with 1 ml PBS and treated with cell culture lysis buffer (Promega, Madison, Wis.). Luciferase quantification was performed using a Luciferase assay system (Promega) on a Luminometer from Dynex Technologies, Inc. (Chantilly, Va.). The amount of protein in the cell lysate was determined using a standard curve of bovine serum albumin (Sigma, St. Louis, Mo.) and a BCA protein assay kit (Pierce, Rockford, Ill.) (n=4).

Cell Viability Assay.

Cells were plated in 24-well plates and transfections were carried out when cellular confluency reached approximately 70%. Polyplexes were prepared as they were for the Luciferase reporter gene assay. Respective cell cultures were transfected in the presence of serum with the addition of 20 ml equilibrated polyplex in HEPES buffer solution (0.5 mg pDNA) to each well. Cells were left to incubate for a total of 18 h before analyzing cell viability using an MTT assay (Sigma). Percent cell viability was determined relative to untreated cells (n=4).

### Results

1. Two-step Synthesis and Characterization of p(TETA/CBA)5k

Synthesis and Characterization.

p(TETA/CBA).

p(TETA/CBA) has been proven as a highly effective gene carrier, and it can derive a variety of branching structures the engineer hyperbranched architecture with no significant cell toxicity. The samples were synthesized and purified as shown in Scheme 1 for subsequent testing. Polymerization occurs via Michael addition of the CBA monomer to the amines present in the TETA monomer. Because four reactive amine groups exist on the TETA monomer, highly branched products can be obtained prior to their gelation. Polymerization reactions were carried out at different temperatures in 100% MeOH and monitored by ¹H NMR. Synthesis temperature was shown to correlate with the degree of branching in each sample (data not shown). Eliminating oligomer polycations from the sample with 1 kDa, 5 kDa, or 10 kDa MWCO ultrafiltration membrane reduced the sample polydispersity index (PDI) as expected, which further correlates with relative molecular weight of the sample when monitored using FPLC. Commercial bPEI25k was also analyzed as an external control for comparison. Because the Mn and Mw values for bPEI25K are underestimated using GPC analysis, extrapolations need to be made to estimate poly(TET/CBA) molecular weight. Moreover, p(TETA/CBA)5k has a similar buffer capacity to the sample obtained by following the original purification approach (Table 1).

TABLE 1

| | | | | | Degree^{c} |
|---|---|---|---|---|---|
| | Mₙ | M_{w} | PDI | Titration^{b} | Branch- |
| Sample | (kDa)^{a} | (kDa)^{a} | (M_{w}/Mₙ) | (μmol HCl) | ing |
| p(TETA/CBA)1k | 4.2 | 8.2 | 1.95 | 25.2 | 0.68 |
| p(TETA/CBA)5k | 5.8 | 8.85 | 1.53 | 27.6 | 0.91 |
| p(TETA/CBA)5k- | 8.9 | 10.6 | 1.19 | 22.3 | — |
| g-PEG2k | | | | | |
| bPEI 25 kDa | 16.4 | 21.0 | 1.28 | 32 | — |

^{a}Number average molecular weight (Mn), weight average molecular weight (Mw), an polydispersity (Mw/Mn) determined using FPLC.
^{b}Polymer fraction buffer capacity titrations determined by the mol of HCl required to shift pH from 7.4 to 5.1 in 0.1M aqueous NaCl.
^{c}Degree branching was determined by MALDI-TOF.

p(TETA/CBA)5k-PEG2k.

Pegylation can improve polycationic carrier function in the presence of serum both in vitro and in vivo, which is largely due to polyplex surface charge. Particles with a near neutral surface charge, however, tend to aggregate in solution due to their mitigated ionic repulsion forces. Therefore, there was synthesized a p(TETA/CBA)5k-PEGylated product that could be mixed in conjunction with p(TETA/CBA)5k if necessary to easily control the weight percentage (wt %) of PEG to the p(TETA/CBA) polycation to examine the effects on particle characteristics and functionality with a non-toxic branched polycation as a model system (Scheme 2). PEG conjugation to the polycation was monitored at 400 nm using a UV-VIZ spectrophotometer using a standard curve. Reactions were complete by 16 hrs. mPEG5k and mPEG10k were also conjugated as described earlier, however, these graft copolymers were not able to form nanosized particles or provide transgene expression (data not shown). NMR analysis and comparison of peak AUC suggested approximately 0.96/1 mol PEG:(TETA/CBA)5k and is in good agreement with the AKTA FPLC analysis (Table 1).

Influence of p(TETA/CBA) PDI and Molecular Weight Biological Activity.

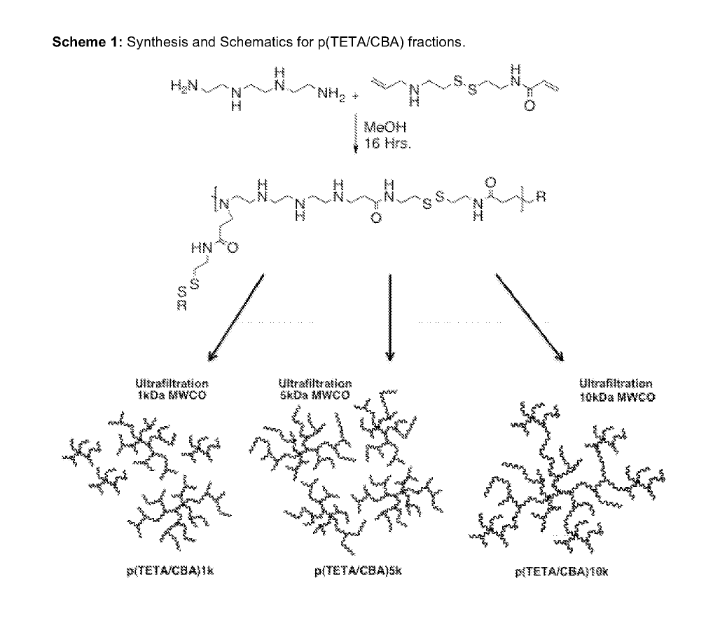

As mentioned previously, LMW PEI exhibits limited pDNA condensation at low N/P ratios and is often perturbed by PEG conjugation, thus, mitigating the PDI of p(TETA/CBA) by eliminating destabilizing oligomers and increasing the average molecular weight without perturbing carrier performance is preferred (17). As seen in FIGS. 1A-D, a reduced p(TETA/CBA) PDI and correlative molecular weight increase has no adverse effects on carrier performance.

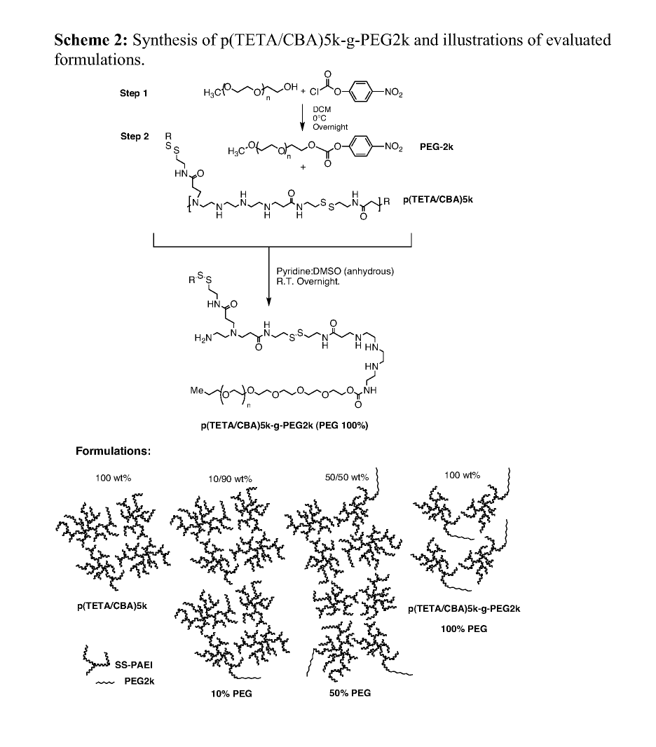

It performs similar to the original synthetic and purification approach for p(TETA/CBA)1k. More specifically, p(TETA/CBA)5k is significantly less toxic in primary HUVEC cells than a current standard bPEI 25 kDa, as well as providing greater luciferase transgene expression in both HUVEC and SVR endothelial cells. This is also true in the case of H9C2 cardiac myoblasts in comparison to p(TETA/CBA)10k (FIGS. 2A-B). The toxicity of bPEI 25 kDa is likely due to the intracellular accumulation of high molecular weight polycationic species (3). These species can interact with and disrupt cell membrane function and/or interact with intracellular proteins and nucleic acids thereby perturbing intracellular and nuclear processes such as cellular trafficking and gene transcription and translation (18, 19). The bioreducible polycation, p(TETA/CBA), most likely mitigates these intracellular interactions and thus toxicity of the primary endothelial cells irrespective of its relative molecular weight, in comparison to the non-degradable bPEI 25 kDa (20). The high transgene expression observed using the p(TETA/CBA) fractions is also likely explained by this phenomenon in conjunction with the intracellular release of nucleic acid (6, 9).

Serum Effects on p(TETA/CBA).

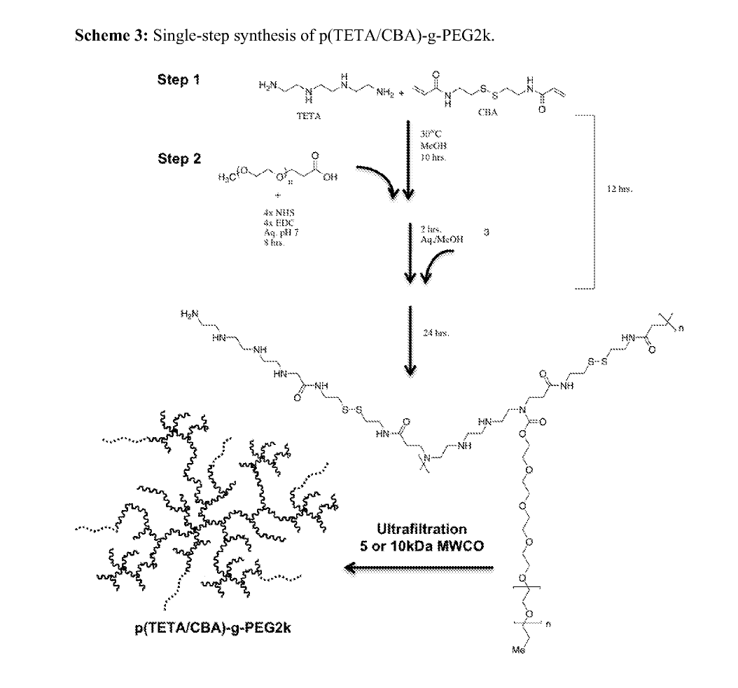

Serum-containing media and serum encountered when polyplexes are administered in vivo often reduces polycationic performance through particle destabilization and nuclease degradation of therapeutic gene or uptake by the reticular endothelial system in vivo. The data presented here are consistent with prior findings. Specifically, p(TETA/CBA) performance on colon adenocarcinoma cells (CT-26) in serum-containing medium is significantly better than bPEI 25 kDa, however, it is low when compared to transfections performed with no serum present in the medium (FIG. 3), thus providing a need to develop a p(TETA/CBA)5k-g-PEG copolymer for nucleic acid delivery as shown (Scheme 2).

Polyplex Characterization.

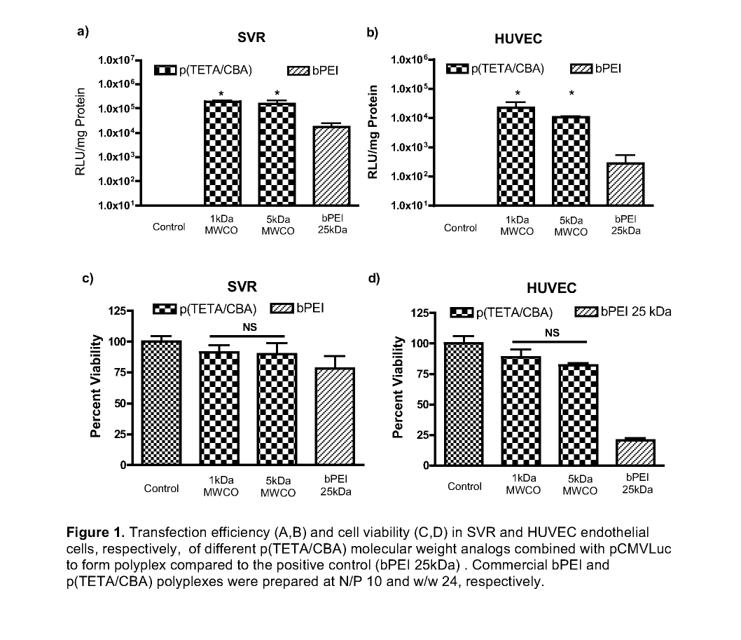

The ability of p(TETA/CBA)5k and p(TETA/CBA)5k-g-PEG2k to form condensed polyplex was investigated by particle size analysis and zeta-potential measurements. Indeed, nanosized particle below or near 200 nm in diameter were formed for both potential gene carriers, however, as expected PEG conjugation interfered with polyplex formation at preferred, low polymer concentrations (FIG. 4A). PEG conjugation did decrease polyplex surface charge at polymer concentrations sufficient to condense pCMVLuc and did not appear to be stable (FIG. 4B).

PEG wt % Effects on Polyplex Characteristics.

Previous findings using PEGylated polyethyleneimine carriers agree with present findings (FIGS. 4A-B) that demonstrate that PEGylation of p(TETA/CBA) polycation disrupts nucleic acid condensation. To overcome this problem and validate the possibility of premixing polymer/PEG-copolymer solutions to control the PEG/polycation wt/wt %, for investigation as well as identify an optimal formulation that maintains homogenous stable polyplex with reduced surface charge, polyplexes were prepared using p(TETA/CBA)5k-g-PEG2k, p(TETA/CBA)5k, and mixtures of the two molecular entities at 10/90 and 50/50 wt/wt %, respectively, at a summed polycation/pDNA w/w ratio equal to twenty-four (Scheme 2).

Serum Stability.

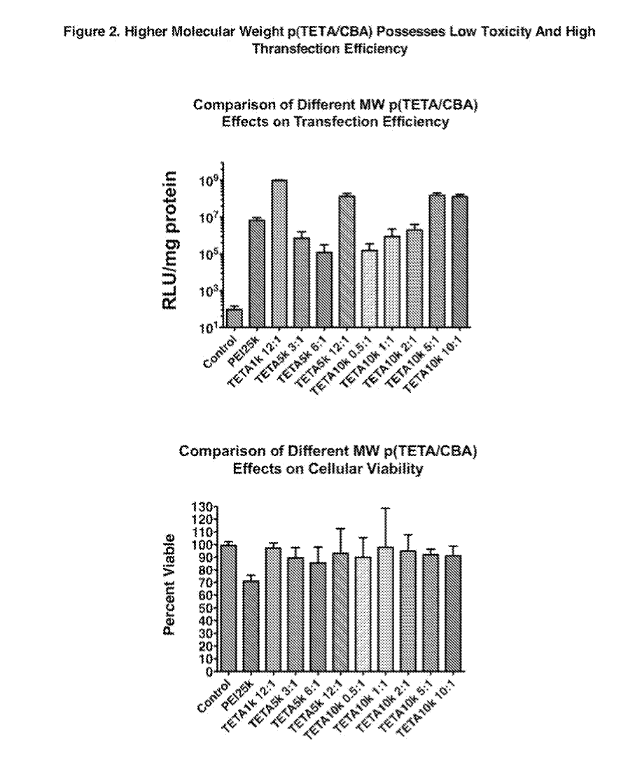

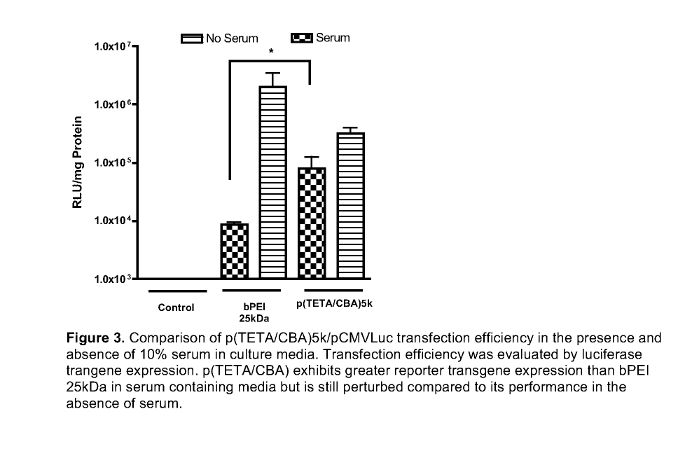

To test the influence of PEG wt % on polyplex stability in the presence of serum, polyplexes were formed and following 30 minutes equilibration were added to fresh rabbit serum to a final serum concentration equal to 90% at 37° C. Aliquots were electrophoresed on an agarose gel to visualize intact pCMVLuc at each time point compared to untreated control at zero hours. FIGS. 5A-B show that p(TETA/CBA)5k and 10% PEG protect pDNA from nuclease degradation to 80% or more at 6 hrs. Increasing PEG wt % to 50 or 100% reduces particle stability and offers less pDNA protection where only 60% and 40% pDNA is preserved, respectively, at 6 h incubation time.

Polyplex Analysis.

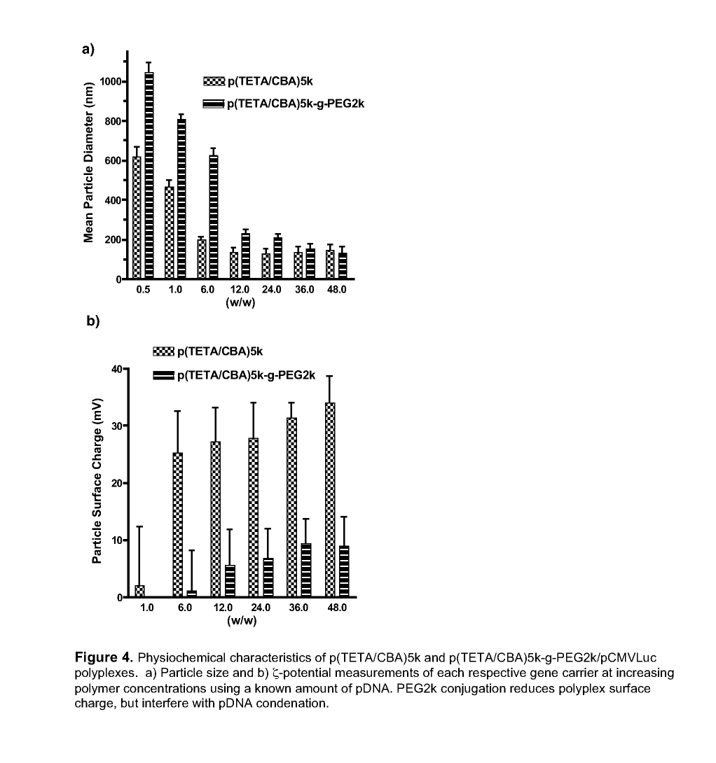

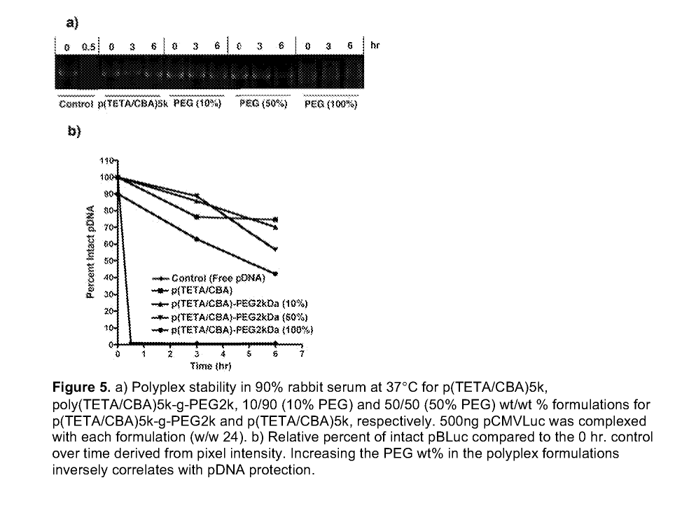

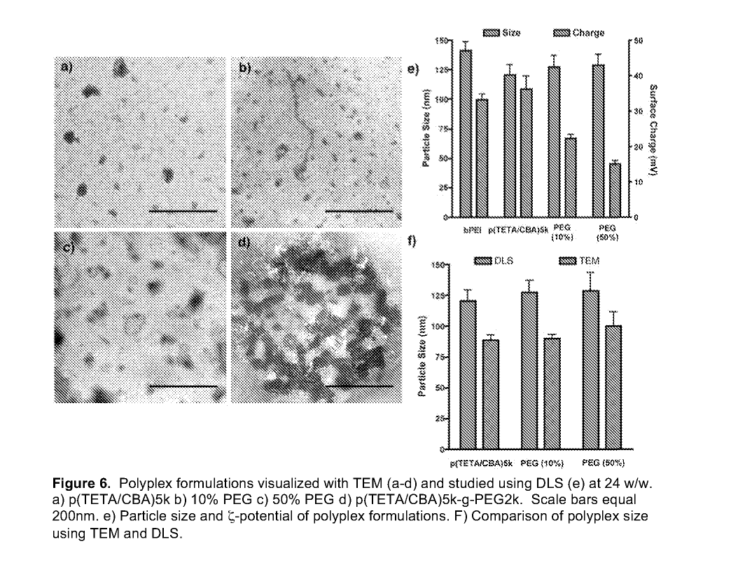

For formulation ease and improved carrier function, stable polyplexes formed using different PEG wt % should display unimodal polyplex size and surface charge with uniform morphology. Polyplex size for each formulation was visualized using TEM (FIGS. 6A-D) and the polyplexes were analyzed to compare their size and distribution to polyplex measurements provided by Dynamic Light Scattering (DLS), which are in agreement with each other and previous findings (FIG. 6F). FIGS. 6A-D reveal morphological changes and less compact polyplexes with translucent outer shells as PEG wt % increases. These translucent outer shells are thought to be from increasing the PEG wt %. p(TETA/CBA)5k-g-PEG2k exhibited aggregation as seen in (FIG. 6D). This aggregation was also noted when analyzed using DLS and adversely influenced the data. Therefore, this formulation is excluded from the analysis and not shown in FIG. 6E. p(TETA/CBA)5k,10 and 50% PEG formulations generate sub-150 nm polyplexes in solution and PEG wt % inversely correlates with polyplex surface charge as expected (FIG. 6E).

PEG Formulations on Carrier Function and Biological Activity.

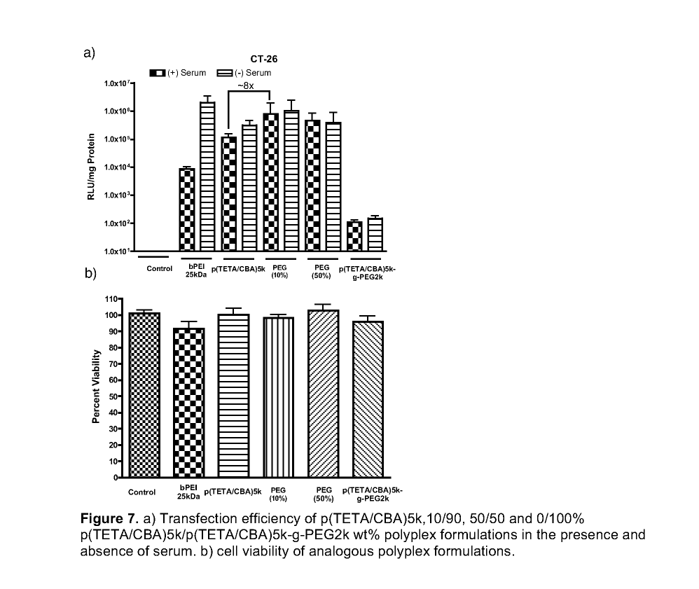

To investigate the potential advantages of PEG-copolymer formulations for gene delivery in the presence of serum a luciferase transgene assay was performed using colon adenocarcinoma cells (CT-26). The 10% and 50% PEG formulated polyplexes exhibited improved transgene expression in the presence of serum compared to the p(TETA/CBA) polycation alone (FIG. 7A). Moreover, these polyplexes are non-toxic to the cells (FIG. 7B).

2. Single-Step Synthesis of p(TETA/CBA)-g-PEG2k

Traditional pegylation synthesis requires steps that are time consuming as two synthesis and filtration steps are required for the polymer product (see Schemes 1 & 2). Therefore, a single step synthesis/filtration method was developed for p(TETA/CBA)-g-PEG2k that reduces the time to final product by half (Scheme 3). The resultant polymer, if purified at a higher molecular weight than previously described (10 kDa MWCO), possesses a broader therapeutic window demonstrated by superior toxicity profiles upon intravenous administration due to a better condensation and protection profile than its lower molecular weight counterparts.

Synthesis and Characterization.

p(TETA/CBA) has previously been proven as a highly effective gene carrier, and it can derive a variety of branching structures for engineering hyperbranched architecture with no significant cell toxicity. The p(TETA/CBA)-g-PEG2k samples were synthesized and purified as shown in Scheme 3 for subsequent testing. Polymerization occurs via Michael addition of the CBA monomer to the amines present in the TETA monomer. As stated previously, four reactive amine groups exist on the TETA monomer, thus highly branched products can be obtained prior to their gelation. This polymer is synthesized by Michael addition of functional amines containing primary and secondary amine moieties to the acrylamide functional group of CBA (1:1 molar ratio). The polymerization is conducted in light sensitive flasks using MeOH as a solvent at 30° C. for 10 hrs under nitrogen atmosphere. Briefly, a brown reaction vessel equipped with a stir bar is charged with TETA and CBA (1M). The vessel is closed and placed in an oil bath set at 30° C. The polymerization is allowed to continue for 10 hr at which time mPEG2k at 10% weight is added dropwise to the reaction after it has been activated with NHS and EDC for 8 hrs in aqueous solution, pH 7. The reaction is then allowed to proceed for two additional hours, at which point 100% excess TETA is added to terminate the reaction. The reaction is then allowed to proceed for an additional 24 hrs to ensure all free acrylamide groups are quenched. The resulting polymer product is isolated by ultrafiltration (MWCO 5000 or 10000) by first diluting the reaction with ultra pure deionized water adjusting to pH 7. Purification is allowed to go overnight at 4 Barr followed by concentration and lyophilization. The ¹H NMR analysis results demonstrate a PEG2k/p(TETA/CBA) ratio of 9% for the 5 kDa filtered polymer and 3-4% or 1 PEG unit per every 146-171 CBA for the 10 kDa filtered polymer. MALDI-TOF analysis demonstrates 91% of the polymer as branched, while 84.3% of the 10 kDa filtered polymer is branched. All PEG appear to be grafted to the 0 arm of the polymer. AKTA FPLC analysis indicates that the p(TETA/CBA)-g-PEG2k filtered at 5 kDa has a mean weight of 10.89 kDA but it had a wide distribution from 3 kDa-30 kDa.

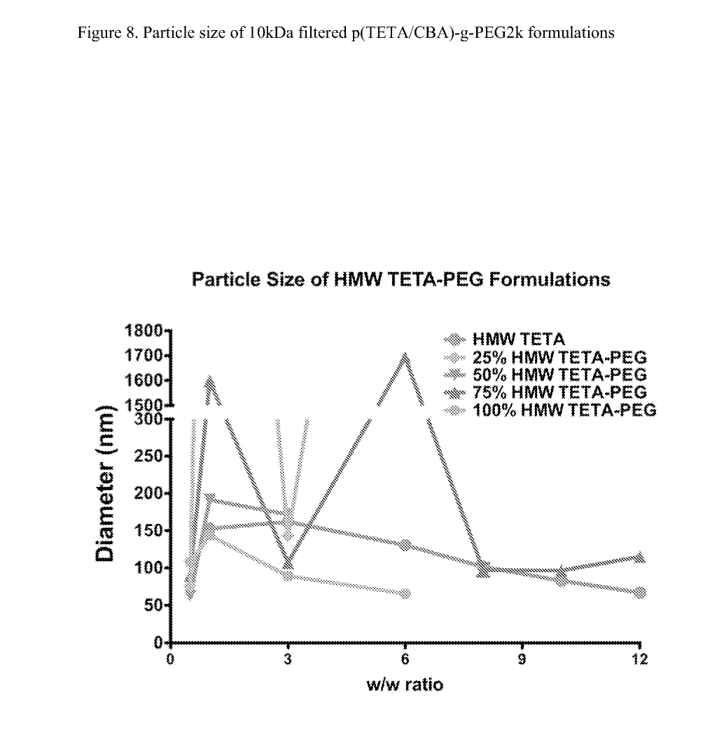

The p(TETA/CBA)-g-PEG2k has similar size characteristics to its two-step synthesis analogue, but the 10 kDa filtered product has smaller complexes at a much lower weight % ratio (FIG. 8 and FIG. 4A).

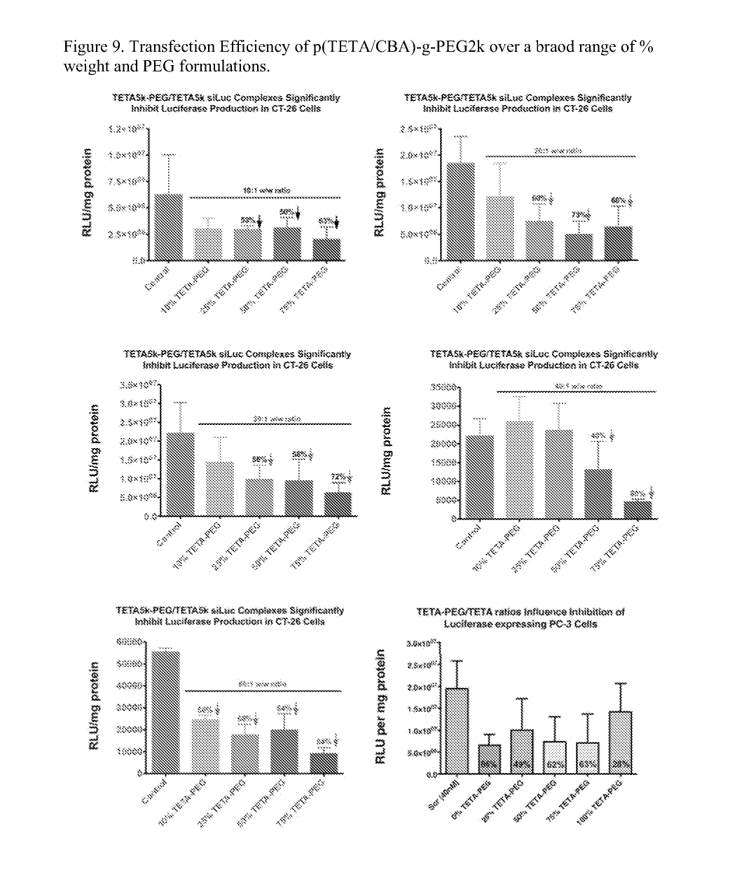

All formulations of p(TETA/CBA)-g-PEG2k have demonstrated excellent transfection characteristics as siRNA carriers. The polymer has a broad range of % PEG formulations and weight % ratios that may be used (FIGS. 9A-F). The polymer was mixed with p(TETA/CBA)5k and complexed to siRNA targeted to luciferase at 40 nM concentration. FIG. 9F shows the polymer working in PC-3 cells. Of note, is that the 100% formulation of p(TETA/CBA)-g-PEG2k was able to inhibit luciferase albeit, at a third lower amount.

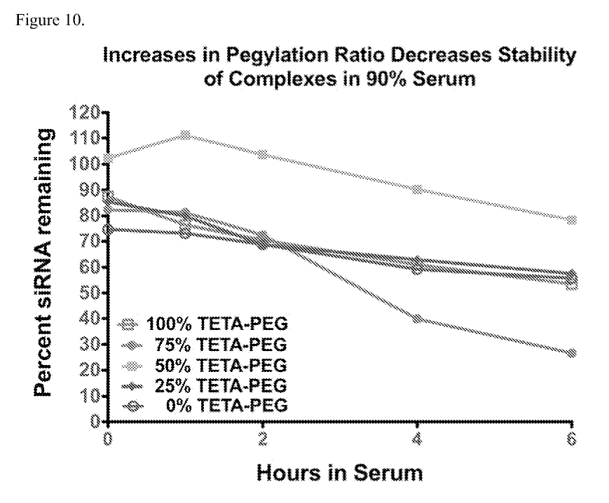

Serum stability of the pegylated polymer formulations was examined in 90% fresh rat serum and examined at 2 hr increments for up to 6 hrs. The siRNA degradation was inhibited best by mixtures of 50% p(TETA/CBA)-g-PEG2k and p(TETA/CBA) but demonstrated a 10% loss following 6 hrs (FIG. 10). Other ratios had 40% or greater loss at 6 hrs.

Polymers filtered at a MW of 5 kDa or lower were found to possess toxicity at 160 μg doses regardless of pegylation. Pegylation has been demonstrated to obscure surface charge and complement activation in PEI conjugates, but it is not evident in this case. This toxicity is evident in both the single and dual step synthesis. The maximum dose able to be delivered with this polymer forming viable nanocomplexes (6:1 weight/weight ratio is ˜27 μg of siRNA/DNA) is less than 1.5 mg/kg. This is deemed too low for in vivo use. Therefore, both polymers (p(TETA/CBA) and p(TETA/CBA)-g-PEG2k) were filtered at 10,000 MWCO using a Centricon centrifugation concentrator. The high molecular weight and low molecular weight fractions were collected (supernatant collected in the upper [high MW] and lower portion [low MW] of the concentrator) and used for characterization. The low molecular weight fraction did not complex well when mixed at weight to weight ratios below 10:1 and had high particle sizes (1200 nm) even at much higher weight to weight ratios of 24:1.

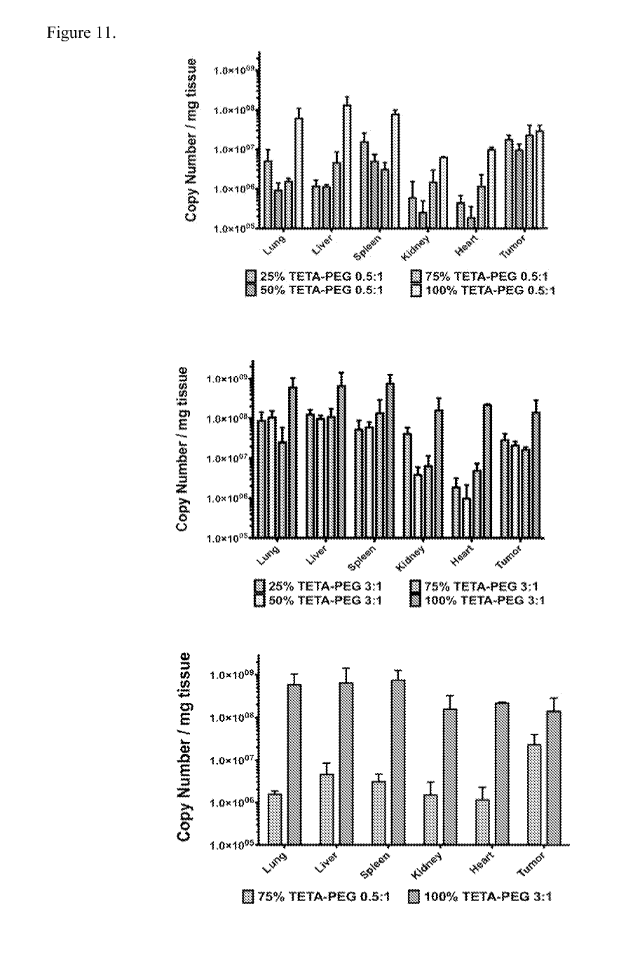

A total of 40 ng of plasmid DNA was complexed by various weight formulations of p(TETA/CBA)-g-PEG2k/p(TETA/CBA) for biodistribution studies. Nanocomplexes were injected intravenously into CT-26 tumor-bearing Balb/c mice via tail vein at 25, 50, 75, and 100% p(TETA/CBA)-g-PEG2k weight formulation ratios in 200 μl of 20% glucose 10 mM HEPES. The animals were sacrificed 48 hrs later, organs (and tumor) extracted, and plasmid DNA was analyzed by qPCR using Taqman primers directed at the F1 ori region of the plasmid. Biodistribution pattern results indicate that maximum gene delivery to the tumor was obtained by a 3:1 polymer to pDNA ratio using 100% p(TETA/CBA)-g-PEG2k (FIG. 11A). However higher levels of plasmid DNA was evident at multiple other tissues. This biodistribution trend was also evident in other % p(TETA/CBA)-g-PEG2k polymer formulation mixtures using the same polymer weight/pDNA weight mixtures but at lower values. A 0.5/1 polymer weight/pDNA weight mixture demonstrated a different biodistribution pattern (FIG. 11B). Tumors demonstrated high levels of plasmid DNA in relation to other tissues with the most difference seen in a 75% p(TETA/CBA)-g-PEG2k formulation. As the biodistribution patterns were the same for the polymer/pDNA w/w mixtures regardless of % p(TETA/CBA)-g-PEG2k formulations one formulation mixture was picked from each to represent the group (FIG. 11C).

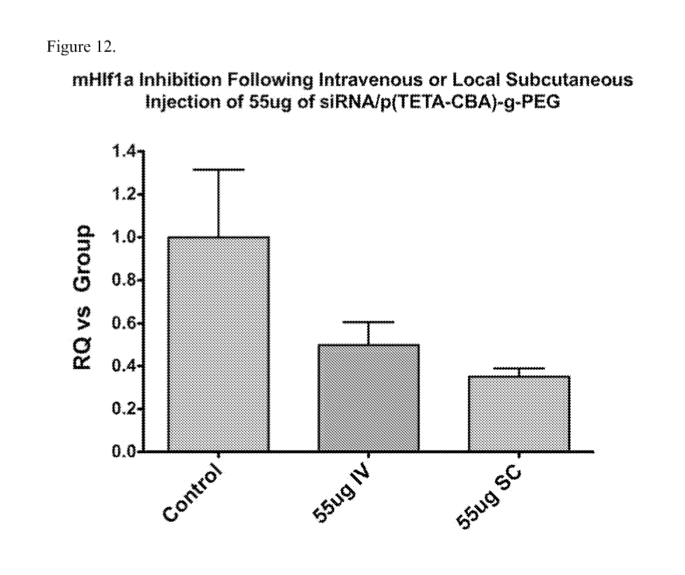

The maximum dose of the nanocomplexes is limited by precipitation, physical forces (hydrodynamic effect), and dose-limiting toxicity therefore, the maximum dose that can be given is currently believed to be 55 μg of siRNA at a 3:1 polymer weight/siRNA weight ratio in 275 μl volume of 20% Glucose and 10 mM HEPES. Nanocomplexes were injected intravenously into CT-26 tumor-bearing Balb/c mice via tail vein or locally (tumor site) at 75% p(TETA/CBA)-g-PEG2k weight formulation ratios at 0.5/1 and 3/1 polymer(s) to mouse HIF-1a targeted siRNA. The mice were sacrificed and organs, and tumor collected from each. Total RNA was isolated using a SV96 Total RNA purification kit and mRNA values were compared among control mice receiving a 20% glucose 10 mM HEPES injection, i.v. and local injections using RT-qPCR. Preliminary Comparative Ct RT-qPCR revealed a 63% and 70% reduction in mHIF-1a values at the tumor site of intravenous and local injection animals, respectively (FIG. 12).

The synthesis for p(TETA/CBA)-g-PEG2k according to the present invention is an improvement over previous methods using bioreducible molecules and poly amidoamines (PAAs) or poly amido ethylenimines (PAEIs). The characteristics are similar but the synthesis is 50% faster than conventional methods and produces a different product than the two-step synthesis method. The p(TETA/CBA)-g-PEG2k when purified at 10 kDa using ultrafiltration has better physiochemical characteristics than its 5 kDa filtered counterpart. The 10 kDa polymer has a better toxicity profile in vivo and maintains good transfection efficiency at the tumor site through a deselective targeting most likely provided by the enhanced permeation and retention effect (EPR). The lower molecular weight polymer cannot deliver the amounts required to demonstrate >50% inhibition due to complexation and dose-limiting toxicity issues. Of the % PEG formulations and weight % ratios it appears that the 75% p(TETA/CBA)-g-PEG2k at 0.5:1 w/w and the 100% p(TETA/CBA)-g-PEG2k at 3:1 w/w are the best candidates for intravenous in vivo delivery of siRNA for inhibiting proteins within tumors. Higher weight/weight ratios were tested but exhibit toxicity due to dose-limiting toxicity. In vitro applications may exist at higher weight to weight ratios at different % formulations and should not be dismissed. Mixtures of p(TETA/CBA)-g-PEG and p(TETA/CBA) exhibit a synergistic effect.

### REFERENCES

(1) Saito, G., Swanson, J. A., and Lee, K. D. (2003) Drug delivery strategy utilizing conjugation via reversible disulfide linkages: role and site of cellular reducing activities. Adv. Drug Delivery Rev. 55, 199-215.
(2) Christiano, R. J., and Roth, J. A. (1995) Molecular conjugates: a targeted gene delivery vector for molecular medicine. J. Mol. Med. 73, 479.
(3) Merdan, T., Kopecek, J., and Kissel, T. (2002) Prospects for cationic polymers in gene and oligonucleotide therapy against cancer. Adv. Drug Delivery Rev. 54, 715-758.
(4) Lee, M., and Kim, S. W. (2004) Polyethylene glycol-conjugated copolymers for plasmid DNA delivery. Pharm. News. 9, 597.
(5) Boussif, O., Lezoualc'h, F., Zanta, M. A., Mergny, M. D., Scherman, D., Demeneix, B., and Behr, J. (1995) A versatile vector for gene and oligonucleotide transfer into cells in culture and in vivo: polyethylenimine. Proc. Natl. Acad. Sci. 92, 7297-7301.
(6) Banerjee, P., Reichardt, W., Weissleder, R., Bogdanov, A. (2004) Novel Hyperbranched Dendron for Gene Transfer in Vitro and in Vivo. Bioconjugate Chem. 15, 960-968.
(7) Fischer, D., Bieber, T., Li, Y., Elsasser, H. P. and Kissel, T. (1999) A novel non-viral vector for DNA delivery based on low molecular weight, branched polyethylenimine: effect of molecular weight on transfection efficiency and cytotoxicity, Pharm. Res. 16, 1273-1279.
(8) Luten, J., et al. (2008) Biodegradable polymers as non-viral carriers for plasmid DNA delivery. Journal of Controlled Release. 126, 97-110.
(9) Lin, C., Zhong, Z., Lok, M. C., Jiang, X., Hennink, W. E., Feijen, J., and Engbersen, J. F. J. (2007) Novel bioreducible poly(amido amine)s for highly efficient gene delivery. Bioconjugate Chem. 18, 138-145.
(10) Anderson, D. G., Lynn, D. M., and Langer, R. (2003) Semiautomated synthesis and screening of a large library of degradable cationic polymers for gene delivery. Angew. Chem., Int. Ed. Engl. 42, 3153-8.
(11) Christensen, L. V., Chang C. W., Kim W. J., Kim S. W., Zhong, Z., Lin C., Engbersen, J. F., Feijen, J., (2006) Reducible poly(amido ethylenimine) designed for triggered intracellular gene delivery. Bioconjugate Chem. 17, 1233-40.
(12) Martelloa, F., Engbersen, J. F. J., Ferrutia, P. (2008) Abstracts/Journal of Controlled Release, 132 e1-e18.
(13) Plank, C., Mechtler K., Szoka Jr. F. C., Wagner E. (1996). Activation of the complement system by synthetic DNA complexes: potential barrier for intravenous gene delivery. Hum. Gene Ther. 7(12):1437-46.
(14) Wagner, E., Plank, C., Zatloukal, K., Cotten, M. and Birnstiel, M L. (1992). Influenza virus hemagglutinin HA-2 N-terminal fusogenic peptides augment gene transfer by transferrin-polylysine-DNA complexes: toward a synthetic virus-like gene-transfer vehicle. Proc. Natl. Acad. Sci. USA. 89(17): 7934-7938.
(15) Verbaan, F. J., Oussoren, C., Van Dam, I. M., Takakura, Y., Hashida, M. D., Crommelin, J. A., Hennink, W. E., Storm, G. (2001) The fate of poly(2-dimethyl amino ethyl)methacrylate-based polyplexes after intravenous administration. Int. J. Pharm. 214, 99-101.
(16) Suh, W., Han, S. Yu, L. and Kim, S. W. (2002) An Angiogenic, Endothelial-Cell-Targeted Polymeric Gene Carrier. Molecular Therapy. 6(5), 664-672.
(17) Merkel O. M., et al., (2009) Integrin aᵥb₃ Targeted Gene Delivery Using RGD Peptidomimetic Conjugates with Copolymers of PEGylated Poly(ethylene imine) Bioconjugate Chemistry. 20, 1270-1280
(18) Burckhardt B. C., Thelen P. (1995) Effect of primary, secondary and tertiary amines on membrane potential and intracellular pH in Xenopus laevis oocytes. Pflugers Arch. 429(3):306-12.
(19) Wang, H. et al. (2006). Binding of Sodium Dodecyl Sulfate with Linear and Branched Polyethyleneimines in Aqueous Solution at Different pH Values. Langmuir. 22, 1526-1533.
(20) Godbey W T, Wu, K. K., Mikos A. G., Poly(ethylenimine)-mediated gene delivery affects endothelial cell function and viability. Biomaterials. 2001; 22:471-480.
(21) Lin, C., Zhong, Z., Lok, M. C., Jiang, X., Hennink, W. E., Feijen, J., Engbersen, J. (2006) Linear poly(amido amine)s with secondary and tertiary amino groups and variable amounts of disulfide linkages: Synthesis and in vitro gene transfer properties Journal of Controlled Release 116: 130-137
(22) Kunath, K., Merdan, T., Hegener, O., Häberlein, H., Kissel, T. (2003) Integrin targeting using RGD-PEI conjugates for in vitro gene transfer. The Journal of Gene Medicine. Volume 5 Issue 7, Pages 588-599

The subject matter claimed is:

1. A composition comprising a graft copolymer of poly(TETA/CBA) and polyethylene glycol.

2. The composition of claim 1 wherein the graft copolymer of poly(TETA/CBA) and polyethylene glycol has a structure as represented in Scheme 2 or Scheme 3.

3. A complex comprising a nucleic acid and a graft copolymer of poly(TETA/CBA) and polyethylene glycol.

4. The complex of claim 3 wherein the nucleic acid comprises plasmid DNA.

5. The complex of claim 3 wherein the nucleic acid comprises siRNA.

6. The complex of claim 3 further comprising poly(TETA/CBA) mixed with the graft copolymer.

7. A composition comprising a mixture of (a) poly(TETA/CBA) and (b) a graft copolymer of poly(TETA/CBA) and polyethylene glycol.

8. A method of transfecting a cell comprising contacting the cell with a complex comprising a nucleic acid and a graft copolymer of poly(TETA/CBA) and polyethylene glycol.

9. The method of claim 8 wherein the nucleic acid comprises plasmid DNA.

10. The method of claim 8 wherein the nucleic acid comprises siRNA.

11. The method of claim 8 further comprising poly(TETA/CBA) mixed with the graft copolymer.

12. A method of making a graft copolymer of poly(TETA/CBA) and polyethylene glycol, the method comprising:
(A) mixing TETA and CBA to form a first mixture and causing the first mixture to react for a first selected period of time to result in poly(TETA/CBA);
(B) then adding polyethylene glycol to the first mixture to form a second mixture and causing the second mixture to react for a second selected period of time; and
(C) purifying the graft copolymer of poly(TETA/CBA) and polyethylene glycol.

13. The method of claim 12 wherein the purifying is by ultrafiltration.

14. The method of claim 13 wherein the ultrafiltration comprises a 5 kDa MWCO.

15. The method of claim 13 wherein the ultrafiltration comprises a 10 kDa MWCO.
